(19) United States
(12) Patent Application Publication
(10) Pub. No.: US 2018/0330825 A1
(43) Pub. Date: Nov. 15, 2018

(54) Physician-Patient Active Learning Base Communication Method and System

(71) Applicants: Mehrbod Sharifi, Scarsdale, NY (US); Abbas Rafini, Palo Alto, CA (US)

(72) Inventors: Mehrbod Sharifi, Scarsdale, NY (US); Abbas Rafini, Palo Alto, CA (US)

(21) Appl. No.: 15/978,039

(22) Filed: May 11, 2018

Related U.S. Application Data
(60) Provisional application No. 62/505,120, filed on May 12, 2017.

(51) Int. Cl. G16H 50/20; G16H 80/00; G06F 17/28

(52) U.S. Cl. CPC G16H 50/20; G06F 17/28; G16H 80/00

(57) ABSTRACT

A system intelligently implements communication between at least one patient and at least one medical practitioner, preferably using two-way natural language conversation. The patient discloses or is prompted to disclose relevant medical condition and background, daily routines, that a medical practitioner would want to diagnose and prescribe for a present medical condition. The conversation is content-based routed in real-time to multiple potential medical responders, preferably including a machine learning (ML) based software agent, as well as human responders having various levels of medical expertise. Such routing advantageously minimizes volume of irrelevant information sent to a responder, and also reduces the cost of information responded to by the most appropriate responder. Over time, the ML-based software agent improves performance by using training data from patient-system communications, and/or via active learning methods. Compartmentalization of patient information promotes patient privacy policies while maximizing relevance of patient-system

### PRIORITY CLAIM

Priority is claimed from applicants' pending U.S. Provisional patent application entitled “Active Learning Based Patient-Doctor Communication Platform”, filed 12 May 2017, provisional application Ser. No. 62/505,120.

### FIELD OF THE INVENTION

The invention relates generally to implementing information transfer between medical practitioner(s) and patient(s) to enable medical diagnosis and treatment plan, especially in areas where in-person physician-patient communication is not feasible. More specifically, the invention relates to an intelligent active learning base communication method and system implementing and improving such information transfer, to promote quicker and more accurate diagnoses and courses of treatment

### BACKGROUND OF THE INVENTION

The cost of medical care is very high. Despite the high workload of individual physicians, there is often a considerable time delay before a patient's appointment with a physician, perhaps many weeks. During even a short appointment visit, the physician must make decisions based upon patient presenting symptoms and medical history. Such rapid decision making can be challenging for the physician and may require further visits and tests to arrive at a better diagnosis and course of treatment. Such further visits and tests will increase the cost of the medical care provided. Some patients may have complex symptoms, which will consume a disproportionate fraction of the physician's time, relative to all patients the physician must actually see.

Too often patients in some geographic areas have limited personal access to medical practitioners, e.g., physicians, trained nurses, trained technicians, etc. There may be no qualified medical practitioners in close proximity, or perhaps transportation to such practitioners is not available. Such patients may seek medical advice remotely, e.g., using websites such as www.webmd.com, www.mayoclinic.com. Such patients may use the Internet to view medical forums to try to self-diagnose their medical condition. Several websites such as www.healthtap.com have medical staff on hand to respond professionally to patient medical questions.

Too often, such patients simply will have limited access to qualified medical practitioners, and may have to resort to a calling service, if the desired physician employs such service. The calling service responder obtains information from the patient and, depending on the perceived urgency, a medical practitioner, perhaps the patient's physician, may return the patient's call. However, often there is a time delay between the patient's initial call and a return call from a medical practitioner. Further, the quality of the opinion rendered telephonically may be substantially less than if an opinion were rendered after a more personal medical practitioner-patient interaction. In some medical fields, innovative telemedicine techniques are used, by which medical practitioners may practice medicine remotely with the patient, using consumer voice/video capable devices, perhaps a smartphone.

Generally, the patient's symptoms at the time of the initial visit may carry greater weight in shaping the physician's opinion than if the physician could have examined the patient sooner, when a perhaps more revealing sequence of symptoms was present. Each patient visit may begin from a cold start without receiving the benefit of a preliminary summary of patient history and conditions and possible symptoms. Further, a same set of symptoms may produce different diagnosis or treatment options, depending on which symptom or set of symptoms receives a higher weight in the physician's decision making. Such weighting may be a function of the physician's individual medical training and experience, and may vary from physician to physician.

Further, patients often take too little time to complete medical forms to communicate their complete history to medical practitioners, while the recipient medical practitioners often have insufficient time to process such information. As a result, important and relevant aspects of the patient's medical history may be overlooked. Some medical practitioners may reach decisions based upon personal training or experience, perhaps in the prescription of antibiotics. Some physicians tend to overly prescribe, while other physicians may under prescribe such medication. There also exists a gray area in which such determinations may be erratic as unsupported by sufficient data. In short, there is a need for an ability to normalize medical opinions, which need embodiments of the present invention address.

As noted, current medical practice is almost entirely human driven, which human dependency make medical practice both expensive and not scalable. While emphasis is made upon patient electronic health records (EHR), such records are used primarily for legal and insurance purposes. The contents of EHR provide suboptimal information for the actual patient diagnosis and treatment process. Difficulty is encountered in trying to track and improve the quality of the diagnosis, and treatment result from existing recording keeping. Regrettably, in some areas of medical practice, the lack of complete patient and medical information can result in errors. Errors may also result from certain biases rooted in the art of medicine, which biases may vary from medical practitioner to medical practitioner. Prior art attempts to employ pure algorithmic solutions typically fail to achieve high enough performance to be completely trusted with the sensitive task of promoting faster and accurate medical diagnosis and treatment plans.

In short, prior art human-driven methods of enabling patients to receive timely and preferably accurate medical diagnoses and treatment leave room for improvement. There is need for a perhaps less human-driven mode of communications between a patient in need of medical care and the medical practitioner who must make a diagnosis and prescribe a course of treatment.

The present invention provides an improved communications system and method by which a patient can more rapidly receive competent medical diagnosis and course of treatment.

### SUMMARY OF THE PRESENT INVENTION

The present invention provides an adaptive, intelligent communications interface system for use by patients and medical practitioners, which system promotes a rapid and intelligently directed optimized data flow of natural language medical information. A patient communicates with the system using natural language, perhaps by smartphone or via Internet and a secure website. The overall system includes a computer system, a natural language understanding (NLU) system that provides transparent seamless communication between the patient and the system and medical providers, and machine learning (ML) typically artificial intelligence based decision-making system. The ML system can triage incoming patient information, or perhaps respond directly by eliciting further information, e.g., if the patient reports a fever, the ML system can inquire using natural language, “how high is the fever”, “how many hours have you had this fever”. The ML system can determine an appropriate level of human responder preferably ranging from nurse to physician specialist. If the ML system determines a medical emergency appears at hand, the patient can be instructed to immediately call emergency, e.g., dial 9-1-1.

The ML system can put the patient in rapid contact with at least one of a preferably tiered or layered selection of medical practitioner responders, ranging from perhaps a medical technician, a skilled nurse, to a general practitioner physician, to a specialist physician. The ML system and any of such human responders can access a knowledge base sub-system, with access also to a library of texts, and web-based information. Also accessible to the ML system and the potential human responders is the patient's medical history. If need be, such human responder can put the patient in contact with a third party responder, perhaps one in relative proximity to the patient location. Any such third responder will, with patient permission, be given access to the current state of the patient's medical history, condition, etc. Preferably, patient identity is anonymized within embodiments of the present invention.

Embodiments of the present invention preferably implement machine learning based approach to reduce time and over reliance upon human medical practitioner involvement, while enhancing quality of diagnosis and medical care. The system preferably adopts a layered model of human medical practitioner expertise, and takes into account utility of the system function vs cost of the data being acquired. The strength and advantages of machine learning are integrated with the expertise of medical practitioners in a manner that is transparent to the patient.

Other features and advantages of the invention will appear from the following description in which the preferred embodiments have been set forth in detail, in conjunction with their accompanying drawings.

### BRIEF DESCRIPTION OF THE DRAWINGS

FIG. 1 depicts an exemplary system providing enhanced physician-patient information communications data flow, according to embodiments of the present invention;

FIG. 2 is a flow chart exemplifying intelligent incremental escalation of nature and quality of response to patient communications within the system of FIG. 1, according to embodiments of the present invention;

FIG. 3 is a flow chart exemplifying intelligent active machine learning cycles within the system of FIG. 1, according to embodiments of the present invention;

FIG. 4 is a block diagram exemplifying the role of a connector system, according to embodiments of the present invention;

FIG. 5 is a block diagram exemplifying adaptive model training, according to embodiments of the present invention; and

FIG. 6 is a block diagram exemplifying incremental responder escalation, according to embodiments of the present invention.

### DETAILED DESCRIPTION OF THE PREFERRED EMBODIMENTS

As will now be described, embodiments of the present invention provide a method and system to achieve a good balance between human-driven and algorithmic analysis as applied to the modern practice of medicine.

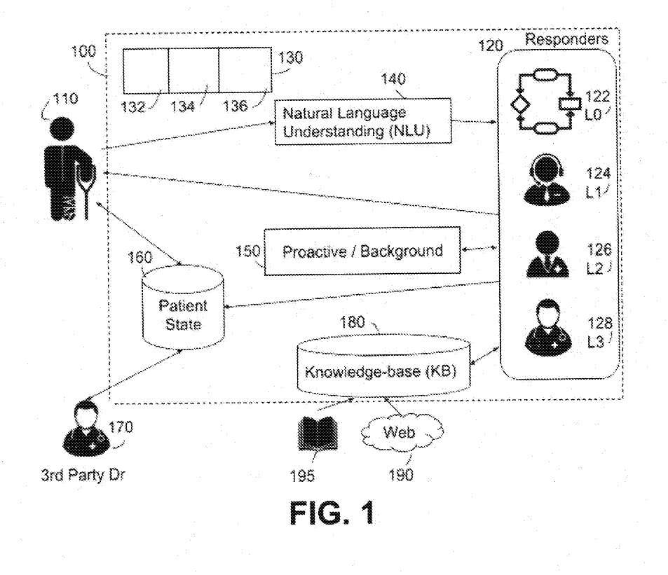

FIG. 1 depicts a system 100 according to the present invention that provides an intelligent adaptive interface between at least one patient 110, who typically wishes to communicate medical information, perhaps “I think I am sick”, to a tiered or layered responder system 120 that includes at least one human medical practitioner. Communications to and from system 100 and the patient are preferably in natural language and may be spoken or input as text, perhaps using a smartphone or computer device via a secure internet website.

In FIG. 1, responder system 120 preferably includes a conversation engine based on machine learning (ML) algorithms at lowest layer responder (L0). Specific algorithms used can be Convolutional Neural Nets (CNN) and Logistic regression (LR) for predictions and sequential models such as Recurrent Neural Nets (RNN) and Long short-term memory (LSTM). Responder system 120 further includes at least a first level (L1) human responder 124, perhaps a medical technician, a second level (L2) human responder 126, perhaps a trained physician assistant or nurse, and a third level (L3) human responder 128, typically a physician, perhaps a medical specialist. Of course there can be more than three tiers of human responders, and more than one human responder at each layer. It will be appreciated that responders 124, 126, and 128 have respectively higher levels of medical qualification.

As shown in FIG. 1, system 100 includes a computer system 130 having a CPU 132, memory 134, and software 136 that is executable by CPU 132 to implement aspects of the system. In some embodiments, system 100 may be a networked or distributed computer system. Computer system 130 can communicate and govern functionality of the various components of overall system 100 shown in FIG. 1.

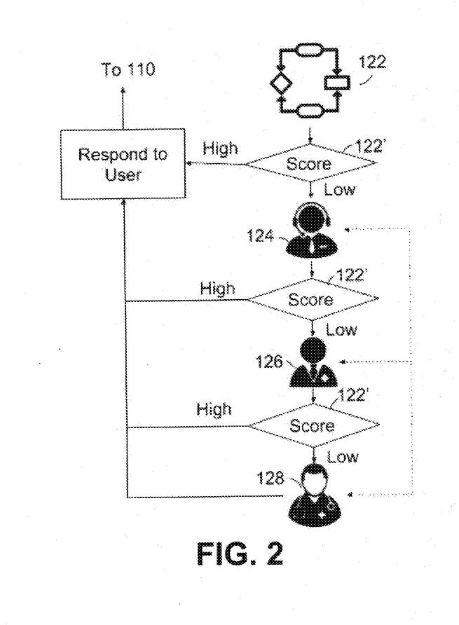

According to embodiments of the present invention, appropriate human medical practitioner expertise input information (e.g. From L1, L2, L3) preferably is utilized by system 100 only when the cost benefit of such level of expertise input is justifiable. The analysis and decision of which layer of medical responder, if any, should respond preferably is carried out by ML system 122, under command of computer system 130. As described later with reference to FIG. 2, preferably a decision scoring portion of ML system 122, denoted system 122′ in FIG. 2, analyzes the patient's incoming communication and using artificial intelligence decides whether a first response should come directly from ML system 122, or from a human responder 124, or 126, or 128. Scoring by ML system 122′ preferably is based on confidence in the patient analysis model under consideration by system 100, and associated risk assessment.

System 100 preferably includes a module that runs in the background of the conversation which may engage responders or patient for additional information in a proactive manner, i.e., without it being initiated from the inquiries. This is designated in FIG. 1 as proactive/background sub-system 150 that may be implemented as part of memory 134. Communications with system 100 may be initiated by patient 110, or may in fact be initiated by the system itself, specifically by proactive/background sub-system 150. For example if patient 110 has previously informed system 100 about a headache, proactive/background sub-system 150 may follow-up, perhaps a day or so later, with an inquiry in spoken English (or other language the patient may have elected to use) as to whether the headache has abated, is still present, or seems worse. System 100 preferably includes a patient state sub-system 160, which may be stored in memory 134. System 100 acts to constantly update patient state sub-system 160 with new information from or to patient 100. If patient 110 responds to inquiry from proactive/background sub-system 150 that the headache has not abated, this new information is stored in patient state sub-system 160, whose contents are available to proactive/background sub-system 150, and vice versa. ML system 122 can determine whether to escalate and bring attention of this on-going system to a higher level responder, perhaps 124. This decision can take into account this patient's past history and other factor such as other current or past condition of other family members. If escalated, responder 124 can make inquiries to the patient. If the headache symptom indeed seems serious, responder 124 can elevate the matter to responder 126 or responder 128. ML system 122′ may determine that incoming symptoms or complaint from patient 110 appear to be a genuine medical emergency, perhaps after consulting a knowledge base 180, and/or the patient's proactive/background sub-system 150 and/or patient state sub-system 160. In such determination, ML system 122 makes the decision to so inform and advise patient 110 to immediately dial 9-1-1 and summon emergency assistance rather the continuing to engage other layers. This patient condition will be input to proactive/background sub-system 150 and patient state subsystem 160. It is a role of ML system 122 to maintain and store up-to-date data on each patient such that information passed to responder system 120 is always current information. Knowledge base 180 preferably includes memory containing general knowledge of all known medical symptoms, all known medical treatments, known prognosis for known treatments for known medical symptoms. Preferably knowledge base 180 can access information from the web 190, from various medical journals and references 195, and preferably information from all proactive inquiries to responder sub-system 120. In preferred embodiments, with patient permission, contents of patient state 160 may be made available to third party practitioners 170, perhaps a physician in close proximity to patient 110.

FIG. 2 provides more detailed information as to decision making and the incremental tiered nature of responder escalation for system 100 in FIG. 1. Under control of computer system 130, a patient's initial communication is passed to machine learning system 122 and will be graded or triaged using a scoring unit, before being passed to an appropriate level responder within responder sub-system 120.

Initially, all patient 110 communications are passed by NLU unit 140 to ML responder system 122. Scoring module within ML module make a decision at each turn of the conversation whether escalation to the higher level is needed based on a model of confidence in language understanding and risk of the information involved. The risk of the information involved may be determined a priori from medical references. Escalation may also be manually done by responders as they see fit. The funneling effect of responders stack essentially classifies the queries commensurate with the expertise level of responders at each level. Effectively, the volume of queries progressively reduces while the complexity of queries increases as the queries flow down the stack of responders.

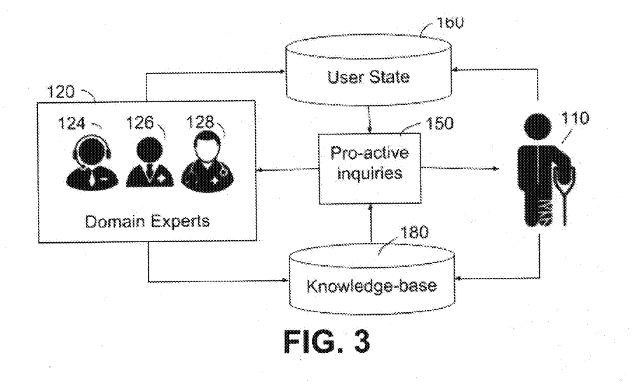

FIG. 3: system makes proactive inquiries from the patient 110 and domain experts responders 120 based on analysis of the contents of user state 160 and knowledge-base 180 in order to increase the coverage of the information within these source. An example for patient side (for updating user state): the system may ask for information about the medical history of the person or his/her family members on occasion when not interacting with patients about symptoms. An example for doctor side (for updating knowledge-base) is when the system discovers that possibly lack of a data point has resulted in some inconsistent outcome for the questions or diagnosis.

It will be appreciated that while performance of system 100 can be significantly improved by using human responders, e.g., 124, 126, 128, the user of software-based systems plays an important role in making quality medical service and advice accessible to more patients. Software system can assist patients in exceptional conditions. For example, if the tone of a patient communication indicates a mishandled past communication, or if human responders, e.g., 124, 126, 128 are not presently available, or there is presently a long response latency in system 100, software system. Maintaining privacy of patient confidential information is important in the medical industry. In general, patient privacy policies are a set of rules that are enforced at each level of service based on prior agreement between the patient and the service provider. The privacy agreement governs different levels of anonymity that the patient has agreed to and the service provider has advertised to maintain at different levels of service. For example, at a so-called service L1, the highest degree of patient privacy is to be maintained.

Within system 100, patient privacy is maintained. As such, patient data including patient medical history, and even attributes such as patient gender and age may remain anonymous. However some ambient information about the patient such as gender or age can become available during patient communication, for instance, from the patient's voice. However, patient-system 100 communication at least at the initial level is mostly driven by the history of the current communication session and statistical data that may be available in knowledge-base 180. In practice, most patient communications are resolved at the low, most basic, L0 responder 122 level, most patients can receive medical advice from system 100 in a patient-anonymous mode.

In some instances, deeper levels of confidential information about the patient and patient symptoms may be required, depending upon the nature complexity level of medical service that may be provided. For example, system may query a female patient about her pregnancy status. But even at this level of service, anonymity preferably is preserved, e.g., by not requiring patient name, address, exact date of birth or social security number. By contrast, all such data are commonly required in typical Electronic Health Record (EHR) platforms.

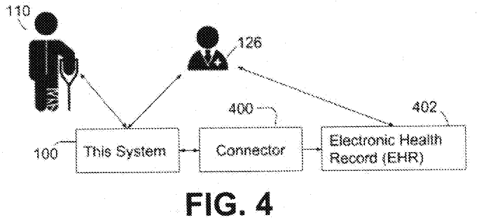

Embodiments of the present invention preferably use a connector sub-system shown in FIG. 4. When it is necessary for system 100 to interface with an EHR 402, perhaps when importing patient communication data. Preferably connector subsystem employs public key cryptography to map the patient's ID internal to EHR from the system 100 to an identifier in the EHR, which mapping is then used to join the patient data. Thus, the system 100 optionally provides such one-way data flow from the EHR without need to include any personally identification information specific to the patient that the physician 126 might need for certain part of the medical practice such as billing purposes.

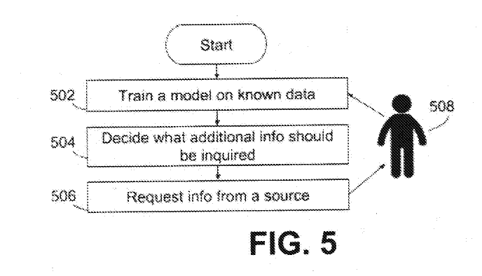

FIG. 5 provides details into how an Active Learning (AL) based system functions in order to collect more information and add it to the network. The selection and integration of new information is driven by statistical rules derived from the set of data that system has seen, overall and for this patient, and also additional knowledge sources it is utilizing. The objective function that this approach optimizes reaching highest increase prediction accuracy using the minimal amount of data inquired (referred to as “minimizing sample complexity”). 502 is a common step in an ML-based system: give a set of known data (e.g., cold frequently shows a symptom of runny nose), we create (called “training”) a model which is a mathematical abstraction of how the patterns in data can be utilized to perform some prediction. For example, showing a symptom of runny nose increase the odds of diagnosis being cold by X %. AL provides a principled approach to acquire additional information when necessary to improve the performance of the model in the prediction task. The core assumption in AL is the availability of the source of ground truth information (called “label”). 504 includes a number of different approaches (e.g., uncertainty sampling) to identify what piece of information should be requested in 506 from the information source 508 (also called “oracle” in literature). In the embodiments of the present invention, responders act as set of oracles.

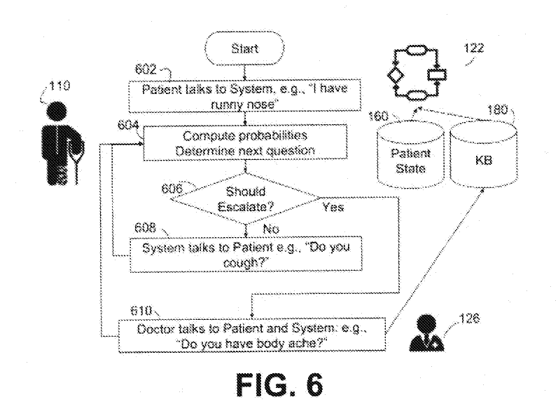

FIG. 6 provides an example flow of the system 100 from FIG. 1 to demonstrate how Active Learning is applied. In this example, step 602, patient 110 tells system 100 “I have runny nose”. At this stage, ML module in L0 is engaged in handling the conversation. Using the KB 180 and patient state 160, in step 604, system creates a list of candidate diseases such as Cold, Flu, Bronchitis, Pneumonia, and so on. At this stage, L0 does not knows based on information in KB 180 and patient state 160 that there are other questions to ask to narrow down the list of possible diseases. Therefore it decides in 606 not to escalate to the higher layers yet and proceed to 608 where system 100 allows 122 to respond with the question from patient “Do you have coughs?”. Response from patient 100 helps 122 to narrow down the list of candidate diseases. For example, if patient said “No” the likelihood of Bronchitis is lowered. As the system proceed with further information collection, perhaps it reaches a point that fails to distinguish between commonly confused Cold and Flu due to lack of information in KB and/or patient state. In that case, step 606 uses scoring mechanism described in FIG. 2 to escalate to higher layers, say for example to physician 126 or L2 who may ask the missing question about body ache in step 610 which helps increasing the likelihood of Flu vs. Cold. Additionally, the missing question is then added to KB 180 to be used in similar situations and effectively reducing the need for escalation in future.

While embodiments of the present invention have been described with respect to patient-medical practitioner interaction communication, it will be appreciated that embodiments may instead be directed to other non-medical fields, customer service for example.

Modifications and variations may be made to the disclosed embodiments without departing from the subject and spirit of the invention as defined by the following claims.

What is claimed is:

1. A system enabling diagnostic communications between a least one patient and at least one medical practitioner provider of medical care, the system including:
a computer system having at least a processor, memory, and at least one software routine stored in said memory and executable by said processor to implement said system;
a responder system, coupled to said computer system, including at least a machine learning (ML) based responder, a first human responder having a first level of medical expertise, and a second human responder having a second, higher, level of medical expertise;
a natural language understanding (NLU) system, coupled to said computer system and to said responder system, said NLU system interfacing spoken communication between said at least one patient and said responder system;
means for triaging medical importance of spoken communication from said at least one patient, triaging coupled to said NLU system and to said responder system;
wherein said means for triaging directs said spoken communication to a chosen responder in said responder system selected from a group consisting of said ML based responder, said first human responder, and said second human responder;
wherein said chosen responder responds to said at least one patient in natural language using said NLU system.

2. The system of claim 1, further including:
memory storing at least a patient state record containing a full medical history of said at least one patient;
wherein said means for triaging is coupled to said memory storing at least a patient state record, and uses contents therein in choosing an appropriate responder from said responder system.

3. The system of claim 1, further including a knowledge base memory, containing general knowledge of at least one of known medical symptoms, commonly selected medical treatments, common prognosis for said known medical symptoms;
wherein said knowledge base memory is accessible to at least one of said computer system, said NLU system, said responder system, and said means for triaging.

4. The system of claim 1, wherein if said machine learning (ML) based responder determines a present complaint by said at least one patient indicates an emergency condition, said system instructs said at least one patient to immediately dial 9-1-1.

5. The system of claim 1, wherein said machine learning (ML) based responder is implemented using artificial intelligence.

6. The system of claim 1, wherein said responder system includes at least one of a human telephone operator, a human licensed nurse, and a human licensed physician.

7. The system of claim 1, wherein said spoken communication is in the form of textual communication

8. The system of claim 1, further including a proactive/background sub-system.

9. The method of claim 1 where the method of active learning is employed to continually improve the MKB to improve the percentage of the conversation handle that exceeds the standard care provided at a medical facility.

10. The method of claim 1 where the user-state database anonymizes patient historic data.

11. The method of claim 1 where the response is of the form of one of at least a reply, a question, an information or an informative piece relevant to the present conversation.

12. The method of claim 1 where information in incrementally collected and processed in an authoritative manner as opposed to patient seeking such information via search engines and forums.

13. The method of claim 3 where the algorithms moderate the conversation with human and augments is with information to reduce time and result in higher reliability in medical domain.

14. The method of claim 3 where at least 90% of conversation is resolved at L0 (ML module).

15. The method of claim 3 where if the conversation is not resolved in any level the conversation escalates to higher layers.

16. The method of claim 3, where L0 is a pure machine learning module

17. The method of claim 3, where L1 is an operator assisted with ML modules, MKB, and user history data.

18. The method of claim 3, where L2 is a medical professional assisted with ML modules, MKB, and user history data.

19. The method of claim 3, where L1 is a medical doctor assisted with ML modules, MKB, and user history data.
